(19) United States
(12) Patent Application Publication
(10) Pub. No.: US 2026/0306783 A1
(43) Pub. Date: Oct. 8, 2026

(54) SYSTEMS AND METHODS FOR REDUCING CO2 REBREATHING FROM A POSITIVE AIRWAY PRESSURE MACHINE

(71) Applicant: SLEEPRES, INC., Murfreesboro, TN (US)

(72) Inventors: William Noah, Pleasant View, TN (US); Bernard Hete, Kittanning, PA (US); Michael Knepper, Friedens, PA (US)

(21) Appl. No.: 19/478,960

(22) PCT Filed: Apr. 25, 2024
(86) PCT No.: PCT/US2024/026260

§ 371 Date: Oct. 27, 2025

Related U.S. Application Data
(60) Provisional application No. 63/462,856, filed on Apr. 28, 2023.
(60) Provisional application No. 63/499,412, filed on May 1, 2023.
(60) Provisional application No. 63/588,467, filed on Oct. 6, 2023.

(51) Int. Cl. A61M 16/22; A61M 16/00; A61M 16/06; A61M 16/08

(52) U.S. Cl. CPC A61M 16/22; A61M 16/0057; A61M 16/022; A61M 16/06; A61M 16/0875; A61M 2205/3334; A61M 2230/40

(57) ABSTRACT

A positive airway pressure machine includes a pressure generator, a mask configured for placement over a patient's face, a conduit operably connecting the pressure generator to the mask, and an exhaust port, A method for reducing CO₂ rebreathing from the positive airway pressure machine includes providing pressure into the patient's airway through the conduit and mask over a treatment period that includes a plurality of inhalation periods and a plurality of exhalation periods in which the pressure provided is at a level sufficient to direct a majority of the patient's exhalation out of the exhaust port. If it is determined that a portion of the patient's exhalation travels into the conduit during an exhalation period, the pressure is augmented to clear the patient's exhalation from the conduit via the exhaust port.

### RELATED APPLICATIONS

This application claims priority from U.S. Provisional Application Ser. No. 63/462,856, filed on Apr. 28, 2023; U.S. Provisional Application Ser. No. 63/499,412, filed on May 1, 2023; and U.S. Provisional Application Ser. No. 63/588,467, filed on Oct. 6, 2023, the entire disclosures of which are incorporated herein by this reference.

### TECHNICAL FIELD

The presently-disclosed subject matter relates to positive airway pressure systems and methods for treating sleep disordered breathing such as obstructive sleep apnea. In particular, the systems and methods of the present invention are configured to reduce the amount of CO₂ exhaled by a patient which is held in the system and subsequently rebreathed during the patient's next inhalation.

### BACKGROUND

Sleep apnea is a potentially serious disorder where breathing repeatedly stops and starts during sleep. Some estimates state that over 25 percent of adults between the ages of 30 and 70 years have sleep apnea, with that number rising to as high as 90 percent in some elderly males.

One of the most common treatments for sleep apnea is continuous positive airway pressure (CPAP) therapy. During therapy, a CPAP machine provides air flow through a circuit, i.e., tubing which terminates with a mask or in-nose nasal pillow connected to the patient. This air flow increases air pressure in the patient's throat, which prevents their airway from collapsing. Subsequent generations of positive airway pressure therapy were designed to reduce pressure at the beginning of the exhalation period (typically between 5-15 cmH2O) in an attempt to improve patient comfort. As there are two different pressures provided, these systems are commonly referred to as bi-level positive airway pressure (BPAP) therapy. Further research and development has resulted in a large array of different positive airway pressure machines but in all instances, the positive airway pressure produces air flow through the machine which interacts with the air flow produced by the patient during inhalation and exhalation. This creates within the circuit a dynamic flow environment. Existing positive airway pressure machines include various valves and ports to control and direct the flow of air through the system. However, there remains a need for an improved positive airway pressure system which provides adequate therapy to a patient while reducing unwanted side effects.

### SUMMARY OF THE INVENTION

The presently-disclosed subject matter relates to positive airway pressure systems and methods for treating sleep disordered breathing such as obstructive sleep apnea. In particular, the systems and methods of the present invention are configured to reduce the amount of CO₂ exhaled by a patient which is held in the system and subsequently rebreathed during the patient's next inhalation.

In some exemplary embodiments of the present invention, a positive airway pressure machine is provided which includes a pressure generator, a mask configured for placement over a patient's face, a conduit operably connecting the pressure generator to the mask, and an exhaust port. In some exemplary implementations, the method of the present invention includes providing pressure into the patient's airway through the conduit and mask over a treatment period that includes a plurality of inhalation periods and a plurality of exhalation periods. The pressure provided is at a level sufficient to direct a majority of the patient's exhalation out of the exhaust port.

In some exemplary implementations, during each inhalation period, the patient inhales less than 0.1% CO₂.

In some exemplary implementations, at the beginning of each inhalation period, none of the patient's exhalation is present in the conduit.

In some exemplary implementations, the method further includes the steps of measuring a volume of the patient's exhalation and adjusting the pressure provided such that the majority of the patient's exhalation is directed out of the exhaust port.

In some exemplary implementations the method further includes the steps of determining that a portion of the patient's exhalation travels into the conduit during an exhalation period and augmenting the pressure to clear the patient's exhalation from the conduit via the exhaust port.

In some particular implementations augmenting the pressure includes increasing the pressure provided at an end portion of the exhalation period.

In some particular implementations augmenting the pressure includes increasing the time during an exhalation period when an increased pressure is provided.

In some particular implementations the method further includes the step of decreasing the pressure provided after the patient's exhalation is cleared from the conduit via the exhaust port.

In some particular implementations augmenting the pressure includes increasing the pressure provided during a beginning portion of the next inhalation period until the patient's exhalation is cleared from the conduit via the exhaust port.

In some exemplary implementations the method further includes the step of providing an initial pressure during a first exhalation period. This initial pressure is configured such that none of the patient's exhalation travels into the conduit. The method then includes the step of decreasing the pressure provided during successive exhalation periods by a predetermined amount until a portion of the patient's exhalation travels into the conduit. The method then includes the step of increasing the pressure to the last pressure when none of the patient's exhalation traveled into the conduit.

In some exemplary implementations the method further includes the steps of receiving data from one or more sensors that measure the pressure or flow through the conduit, and determining whether a portion of the patient's exhalation travels into the conduit.

In some exemplary implementations the method further includes the steps of determining whether the patient is awake or asleep, providing a minimum pressure when the patient is awake, and augmenting the pressure provided over a ramping period of time after the patient is asleep.

In some particular implementations the minimum pressure is sufficient to ensure that, at the beginning of an inhalation period, none of the patient's exhalation is present in the conduit.

In some particular implementations, during the step of augmenting the pressure, the pressure provided at an end of each exhalation period is gradually increased over successive exhalation periods.

In some particular implementations the pressure provided increases during each exhalation period to reach a higher pressure at a period of time prior to the next inhalation period, and during the step of augmenting the pressure, the higher pressure is gradually decreased over successive exhalation periods.

In some particular implementations the pressure provided increases during each exhalation period to reach a higher pressure at a period of time prior to the next inhalation period, and during the step of augmenting the pressure, the period of time is gradually increased over successive exhalation periods.

In some exemplary implementations the pressure is at a relief positive airway pressure during a portion of the inhalation periods including an end of the inhalation period, the pressure is at a therapy positive airway pressure during a portion of the exhalation periods including an end of the exhalation period, the therapy positive airway pressure greater than the relief positive airway pressure, and the pressure decreases from the therapy positive airway pressure to the relief positive airway pressure after the end of the exhalation period.

In some particular implementations the method further includes the steps of determining that a portion of the patient's exhalation travels into the conduit during an exhalation period, and increasing the relief positive airway pressure.

In some particular implementations the method further includes the steps of determining that a portion of the patient's exhalation travels into the conduit during an exhalation period, and maintaining the therapy positive airway pressure for a larger portion of the exhalation periods.

In some particular implementations the method further includes the steps of determining that a portion of the patient's exhalation travels into the conduit during an exhalation period, and increasing the therapy positive airway pressure.

In some exemplary implementations the method further includes the step of modifying the exhaust port to adjust a maximum exhaust flow out of the exhaust port.

In some particular implementations the step of modifying the exhaust port includes replacing the exhaust port with one of a plurality of interchangeable ports, each of the interchangeable ports having a predetermined exhaust flow rate.

In some particular implementations the exhaust port includes an exhaust orifice with an adjustable size and the step of modifying the exhaust port includes changing the size of the exhaust orifice.

In some particular implementations the method further includes the step of providing feedback to a user regarding what modification to the exhaust port is needed.

### BRIEF DESCRIPTION OF THE DRAWINGS

FIG. 1 is a schematic representation of an exemplary positive airway pressure system of the present invention during the inhalation phase of the patient's respiratory cycle;

FIG. 2 is a schematic representation of the exemplary positive airway pressure system of FIG. 1 during a portion of the expiration phase of the patient's respiratory cycle under a first scenario in which the patient's exhaled flow never exceeds exhaust flow;

FIG. 3 is a schematic representation of the exemplary positive airway pressure system of FIG. 1 during a portion of the expiration phase of the patient's respiratory cycle under a second scenario in which the patient's exhaled flow exceeds exhaust flow but is still capable of being cleared from the circuit prior to the next inhalation;

FIG. 4 is a schematic representation of the exemplary positive airway pressure system of FIG. 1 during a portion of the expiration phase of the patient's respiratory cycle under a third scenario in which the patient's exhaled flow exceeds exhaust flow to the extent that it is not cleared from the circuit prior to the next inhalation;

FIG. 4B is a schematic representation of the exemplary positive airway pressure system of FIG. 4 immediately after the beginning of the subsequent inspiration phase of the patient's respiratory cycle;

FIG. 5 is an exploded perspective view of an exemplary face mask capable of use in the exemplary positive airway pressure system of FIG. 1;

FIG. 6 is a detailed view of the exhaust assembly shown in isolation;

FIG. 7 is a process flow diagram of one exemplary implementation of the present invention;

FIG. 8 is a graph of one exemplary applied pressure profile for a positive airway pressure system of the present invention in relation to a graph of a patient's air flow; and

FIG. 9 is a graph of another exemplary applied pressure profile for a positive airway pressure system of the present invention in relation to a graph of a patient's air flow.

### DESCRIPTION OF EXEMPLARY EMBODIMENTS

In most positive airway pressure systems, a patient's exhalation is directed through at least a portion of the circuit before being exhausted into the environment. The exhaled gas can therefore remain in the circuit resulting in the patient rebreathing at least some exhaled gas during the next inhale. When exhaled gas is rebreathed, there are three notable effects: (1) there is increased humidity per breath, (2) there is a reduction in oxygen fraction of the inhaled air, and (3) there is an increase in the fraction of CO₂ of the inhaled air. When experiencing this during positive pressure ventilation, increased humidity might just lead to discomfort for the patient. Reduced oxygen could drop oxygen saturation and result potentially in a cascade of reduced oxygen, but there is a longer time available before oxygen depletion is critical due to the affinity for oxygen of hemoglobin, and the availability, albeit reduced in concentration remaining in the lung from the start of rebreathing. The buildup of CO₂, however, is a major issue due to the fact that it is simply trapped in the circuit and, when it cannot be removed, quickly increases in concentration. Increased levels of inhaled CO₂ cause a variety of dangerous physical and physiologic responses. Increased CO₂ in the air in the lungs reduces the body's ability to remove CO₂ from the blood causing CO₂ to build up in the blood and tissues. As a result, the pH level in the blood decreases which triggers an increased respiratory drive as the body continues to try to remove more CO₂ to return the CO₂ concentration in the blood to normal levels.

The presently-disclosed subject matter relates to positive airway pressure systems and methods for treating sleep disordered breathing such as obstructive sleep apnea. In particular, the systems and methods of the present invention are configured to reduce CO₂ rebreathing during the patient's next inhalation.

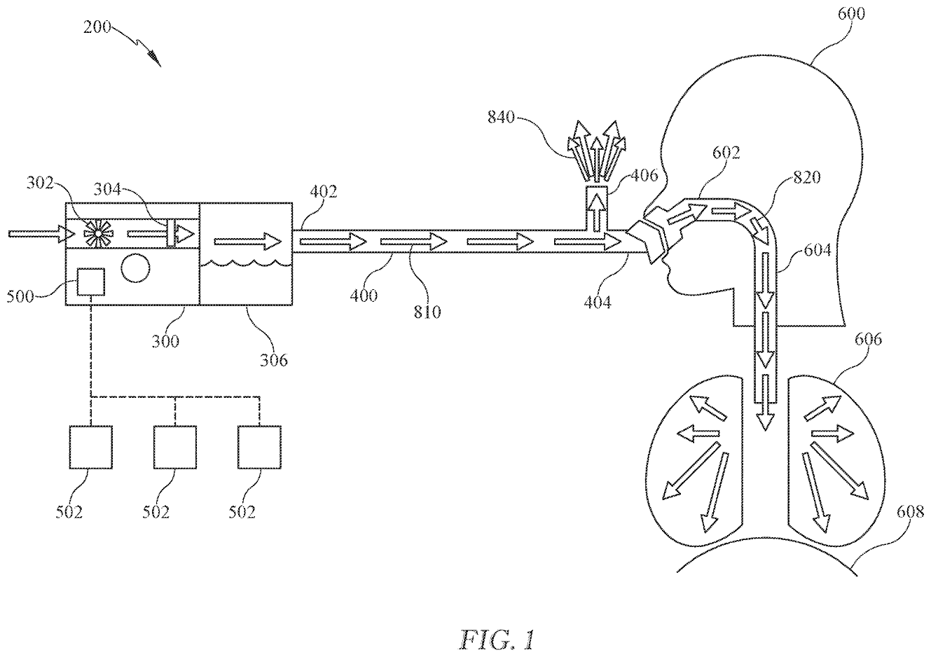

According to some exemplary embodiments, and referring to FIGS. 1-4, a positive airway pressure system 200 of the present invention includes a pressure generator 300 to provide a desired air pressure to a patient 600. Specifically, the exemplary pressure generator 300 includes a fan 302 which draws air in from the environment and through a flow meter 304. The air is then directed through a humidifier 306 before passing from the pressure generator 300 into a conduit 400 which has an inlet 402 operably connected to the pressure generator 300 and an outlet 404 operably connected to the patient's respiratory system as well as an exhaust port 406 along the conduit 400 but preferably near the outlet 404. As discussed further below, in some embodiments, the exhaust port 406 is incorporated into the outlet 404 itself. The pressure generator 300 creates a circuit air flow 810 though the conduit 400, and, as such, the pressure generator 300 can also be referred to as a flow generator. By adjusting the speed at which the fan 302 operates, the pressure generator 300 is able to affect the pressure applied by the system 200 to the patient's respiratory system. Others means of adjusting pressure applied to the patient are possible without departing from the spirit and scope of the present invention. For example, another know means of adjusting pressure is an adjustable valve provided on or near the pressure generator which bleeds off air flow produced by the fan.

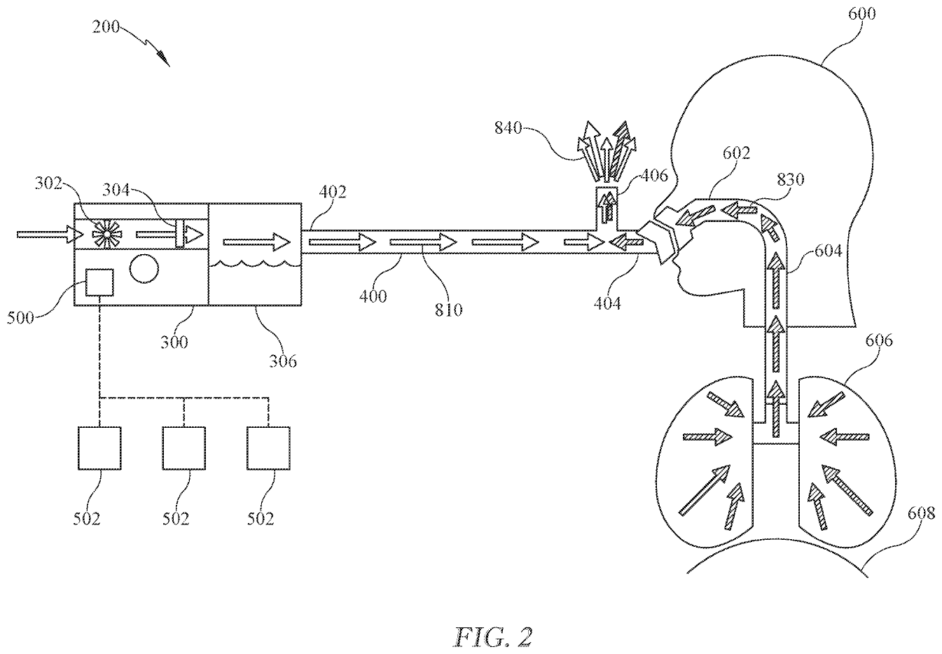

Referring now specifically to FIG. 1, during an inhalation period of the patient's breathing, the diaphragm 608 drops causing the lungs 606 to expand, resulting in an inhalation air flow 820 through the nasal passage 602 and pharynx 604. By comparison, and referring now to FIGS. 2-4, during an exhalation period of the patient's breathing, the diaphragm 608 and lungs 606 of the patient 600 relax resulting in an exhalation air flow 830 out through the pharynx 604 and nasal passage 602. As used herein, the nasal passage 602 is inclusive of the mouth. Also, as used herein, the “airway” of the patient 600 is inclusive of the nasal passage 602, pharynx 604, and lungs 606.

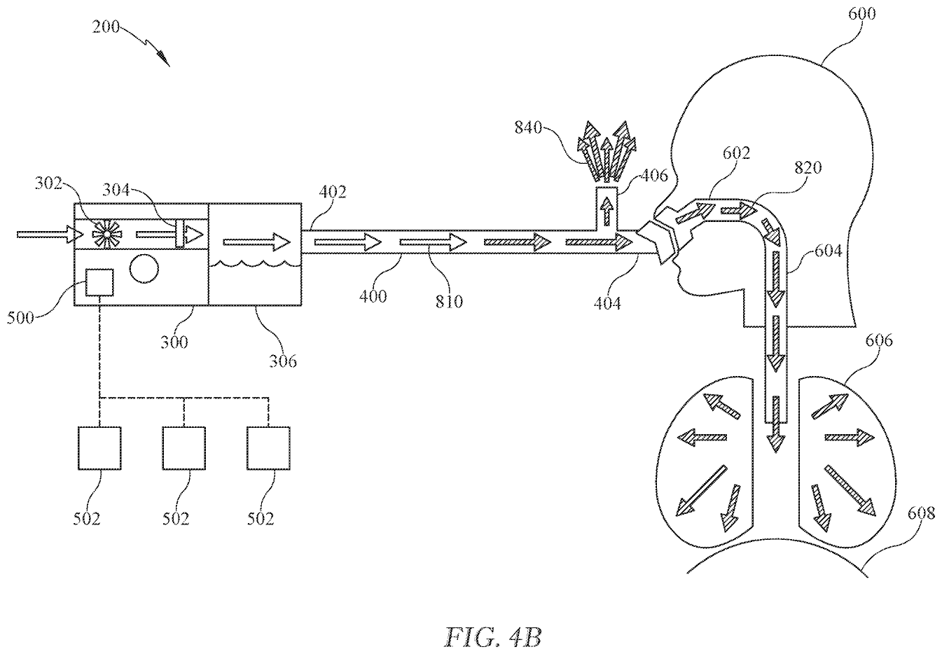

Referring still to FIGS. 1-4, but now also to FIG. 5, one exemplary outlet 404 is in the form of face mask 700 which is secured to the patient's head via a plurality of straps 702. More specifically, the face mask 700 includes a mask body 710 with a seal structure 704 around a periphery of the mask body 710. The seal structure 704 is configured to engage with the face of the patient 600 and provide a seal to prevent, or significantly reduce gas leak around the edge of the mask 700. The exemplary face mask 700 is configured to surround the nose and mouth of the patient 600, but other designs are possible, including masks which only surround the patient's mouth or only surround the patient's nose. To this end, a protrusion 712 extends forwardly from a central portion of the mask body 710 and is shaped to accommodate the nose and the mouth of the patient 600. The protrusion 712 includes an opening 714 through which gas can readily flow in and around the patient's mouth and nose providing for negligible resistance to the inhalation air flow 820 and exhalation air flow 830. The opening 714 is configured to connect with an exhaust assembly 720 that operably connects the mask body 710 to the circuit conduit 400 to thereby provide gas from the pressure generator to the patient's respiratory system. The cylindrical inner surface of the opening 714 in the mask body 710 is shaped and configured to provide a releasable friction fit with a generally cylindrical mating surface of exhaust assembly 720 which itself is removably connected with the circuit conduit 400 for receiving a breathable gas.

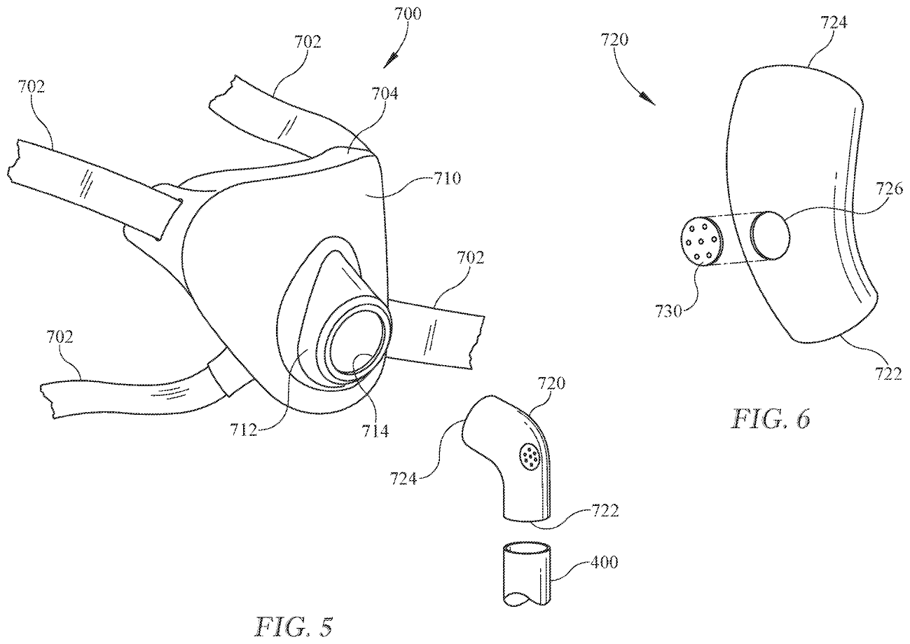

Referring now specifically to FIG. 6, the exemplary exhaust assembly 720 is a generally elbow shaped tubular member formed from a rigid material, e.g., a plastic. The exhaust assembly 720 includes a primary inlet 722, a primary outlet 724, and a secondary outlet 726, which corresponds to the exhaust port 406 shown in FIGS. 1-4. The primary inlet 722 is connected to the circuit conduit 400 and receives pressurized gas from the pressure generator 300. The primary outlet 724 is connected to the face mask 700 and alternately delivers pressurized gas to the patient's respiratory system when the patient inhales and receives the patient's exhalation when the patient exhales, as discussed further below.

The secondary outlet 726 is positioned between the primary inlet 722 and the primary outlet 724 and is configured to exhaust gas from the circuit. To this end, the secondary outlet 726 includes an insert 730 that defines a plurality of orifices through which gas can flow. The exemplary insert 730 is removable and replaceable with other inserts that define a different number, size, or arrangement of orifices to affect the maximum flow of gas out of the secondary outlet 726, as discussed further below. A plurality of interchangeable ports can be provided which each provide a predetermined exhaust flow rate.

Gas that is exhausted through the secondary outlet 726 may include nothing but gas flowing in through the primary inlet 722, nothing but gas flowing in through the primary outlet 724, or a combination of gas from both the primary inlet 722 and the primary outlet 724, as discussed further below. It should be understood that the face mask 700 described above is merely exemplary and additional features may be included without departing from the spirit and scope of the present invention.

For example, in some embodiments, the exhaust assembly 720 may allow for the introduction of gas through an opening other than the primary inlet 722 or the primary outlet 724 when the pressure within the exhaust assembly 720 is below a threshold value. In this way, the exhaust assembly 720, and in particular the secondary outlet 726, may have a secondary function as an entrainment valve without departing from the spirit and scope of the present invention.

The exemplary face mask 700 shown in FIG. 5 is merely one possible implementation of the general outlet 404 shown in FIGS. 1-4 in which the secondary outlet 726 of the exhaust assembly 720 shown in FIGS. 5-6 corresponds to the exhaust port 406 shown in FIGS. 1-4. Furthermore, it is contemplated that, in some embodiments, rather than utilizing a face mask, such as the exemplary face mask 700 shown in FIG. 5, the outlet 404 can be in any suitable form for operably connecting air provided by the pressure generator 300 to the patient, such as, but not limited to, a full face mask, a partial face mask, a nasal mask, a nasal pillow, or any other suitable outlet.

Referring once again to FIGS. 1-4, the exemplary airway pressure system 200 further includes a controller 500 for controlling pressure applied to the patient's airway. The controller 500 includes a computer with a processor for executing instructions stored in a memory component to modulate the pressure generator 300 and pressure applied to the patient's airway. According to some exemplary embodiments, such active control is obtained via an algorithm that controls the device, for example by adjusting the speed of the fan 302, in accordance with the implementations discussed below. The controller 500 is operably connected to a plurality of sensors 502 that measure different aspects of the system 200, the patient 600, and the ambient environment. The sensors 502 can include, for example, a pressure sensor that monitors the pressure of the air in either the pressure generator (e.g., the previously mentioned flow meter 304), the conduit 400, the outlet 404, or the exhaust port 406. The sensors 502 can also include, but are not limited to, a temperature sensor, an ambient pressure sensor, a gauge pressure sensor, a patient flow sensor, ambient humidity sensor, microphone, and accelerometer. External sensors can also be used, for example, to directly measure the patient, including but not limited to respiratory belts, non-contact doppler radar, blood pressure, pulse, and pulse oximeter sensors.

The pressure generator 300 provides pressure through the conduit 400 and the outlet 404 during the inhalation period of the patient's respiratory cycle as well as during the exhalation period of the patient's respiratory cycle. However, depending on the pressure provided by pressure generator 300, the direction of air flow through the conduit 400, outlet 404, and exhaust port 406 varies, as discussed further below.

It is well known and understood that every mask used with a positive airway pressure system possesses some dead volume between the patient and the exhaust port that will fill with exhaled gas that remains at the end of exhalation. The air in this dead volume will therefore be the first air rebreathed during the next inhalation. As used herein, this “deadspace rebreathing” cannot be avoided, as the location of this volume (i.e., between the patient and the exhaust port) precludes it from being flushed out via the exhaust port while the patient is exhaling. Of course, masks typically do not create a perfect seal on the face of the patient and so some of this dead volume is refreshed via unintentional leak. Furthermore, masks can be designed which include features, such as a plurality of small opening or intentionally incomplete seals, which may provide intentional leak to further help alleviate some of this deadspace rebreathing. While this leak, intentional and unintentional, may help somewhat in reducing deadspace rebreathing, it does very little to help clear the patient's exhalation which passes beyond the exhaust port.

When the patient's exhalation passes beyond the exhaust port, another type of rebreathing, referred to herein as “circuit rebreathing,” can occur depending on the pressurized air provided by the pressure generator 300 and the patient's exhalation. There are generally three different scenarios which result in different interactions between the pressurized air provided by the pressure generator 300 and the patient's exhalation with respect to the exhaust port 406, which are illustrated in FIGS. 2-4. Of note, although there is some amount of turbulent mixing of gas flowing through the circuit, it has been found that the flow moves in a generally bolus fashion so that exhaled gas does not substantially mix with fresh air in the conduit.

As shown in FIG. 2, under a first scenario, the circuit air flow 810 and the patient's exhalation air flow 830 both exit through the exhaust port 406 as a combined exhaust flow 840. In other words, the patient's exhalation air flow 830 does not exceed a maximum exhaust flow through the exhaust port 406. In this scenario, no circuit rebreathing occurs as the circuit air flow 810 continues to pass through the entirety of the conduit 400 up to and out of the exhaust port 406 thereby preventing any exhaled CO₂ from lingering in the circuit.

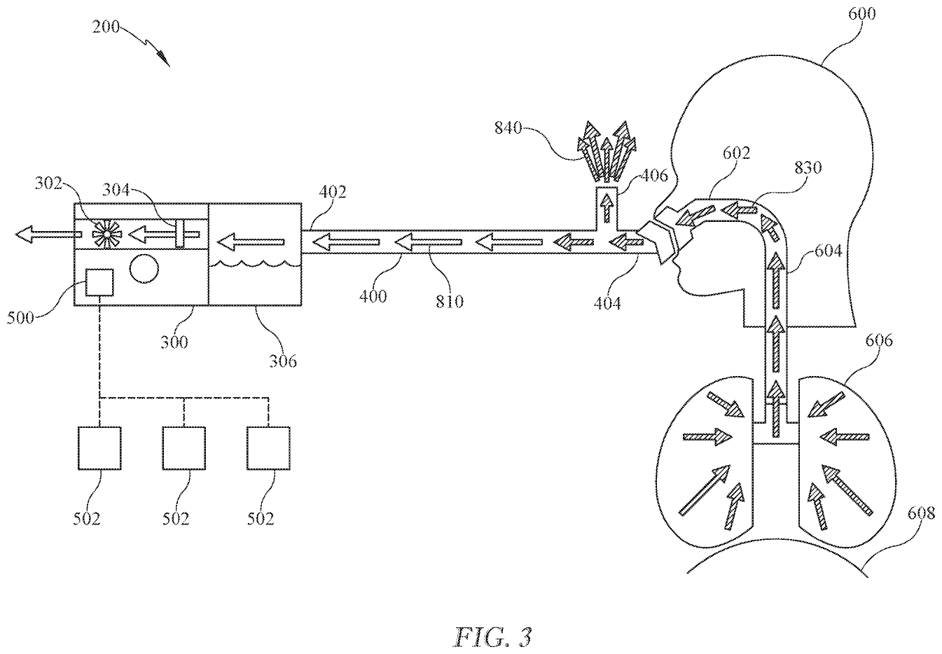

By comparison, as shown in FIG. 3, under a second scenario, the patient's exhalation air flow 830 minimally exceeds the maximum exhaust flow through the exhaust port 406 such that the entirety of the exhaust air flow 840 consists of the patient's exhalation. Excess patient exhalation air flow 830 passes retrograde into the conduit 400. However, there is still no circuit rebreathing in this scenario as the retrograde flow is small enough that the entire volume of the patient's exhalation that traveled into the conduit 400 is exhausted from the exhaust port 406 prior to the beginning of the next inhale as the patient's exhalation air flow 830 approaches zero near the end of the exhalation phase. Of note, because of the bolus air flow mentioned above, when the patient's exhalation air flow 830 passes retrograde into the conduit 400, the direction of the circuit air flow 810 also reverses through the conduit 400 returning into and through the pressure generator 300. Once again, it is understood that the exhaled gas does not substantially mix with fresh air in the conduit.

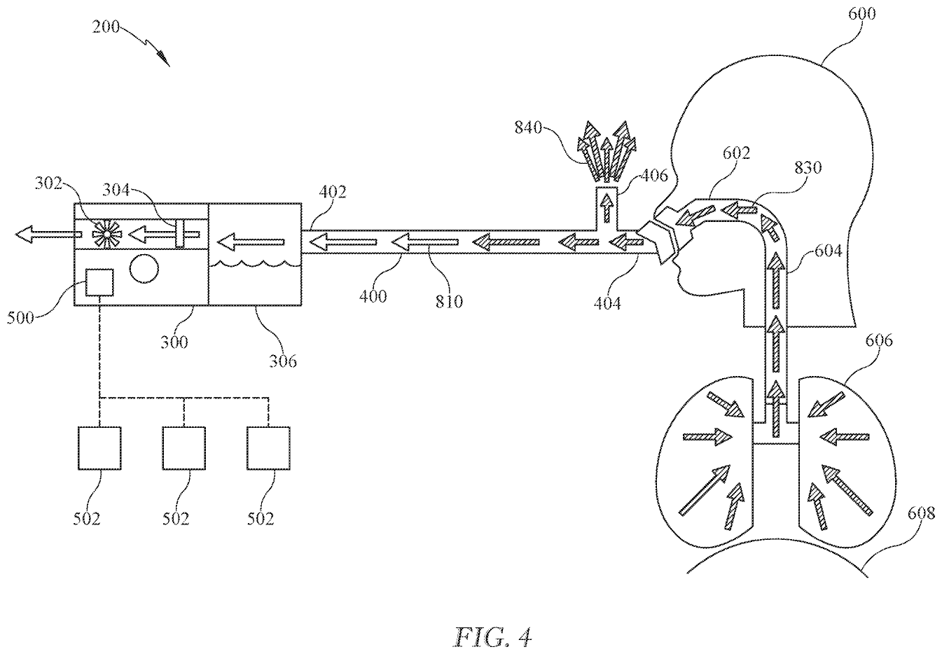

As shown in FIG. 4, under a third scenario, the patient's exhalation air flow 830 greatly exceeds the maximum exhaust flow through the exhaust port 406. Once again, the entirety of the exhaust flow 840 consists of the patient's exhalation but the retrograde flow is so significant that the entire volume of the patient's exhalation that traveled into the conduit 400 cannot be purged from the exhaust port 406 before the next inhalation, and as such, circuit rebreathing does occur. Specifically, and referring now to FIG. 4B, immediately after inhalation begins, the direction of the air flow through the conduit 400 and the patient 600 reverses. The exhaust air flow 840 still consists entirely of the patient's previous exhalation. Due to the large amount of retrograde flow that occurred during the previous exhalation (shown in FIG. 4), there is a sufficiently large volume of excess exhalation contained with the conduit 400 at the beginning of inhalation that at least some of the retained exhalation is inhaled by the patient through at least a beginning portion of the inhalation.

Of note, as shown in FIG. 4B, at the very beginning of inhalation even the inhalation air flow 820 contains elevated levels of CO₂. However, this is true under all of the scenarios shown in FIGS. 2-4 as well as normal breathing. It is only after a patient's airways are cleared of partially exhaled air that the inhalation air flow 820 contains fresh air that is received in the patient's lungs 606, as shown in FIG. 1. Under the third scenario shown in FIGS. 4 and 4B, in order to receive fresh air, not only does the patient's airway need to be clear, but the previously exhaled air retained in the conduit 400, which exceeds what is capable of leaving the exhaust port 406, must also be inhaled before fresh air is received in the patient's lungs 606.

In short, retrograde flow occurs when patient exhalation air flow 830 is greater than the maximum exhaust flow capable through the exhaust port 406 and thus the patient's exhaust flow passes into the circuit back toward the pressure generator 300. Circuit rebreathing will then occur if the volume of retrograde gas flow of the patient's exhalation beyond the exhaust port 406 is too large to be exhausted through the exhaust port 406 before the next inhale. The amount, or severity, of rebreathing is therefore correlated to the volume of the patient's exhalation contained within the circuit at the beginning of the next inhalation.

There is another theoretical fourth scenario in which patient exhalation flow is so high that it not only goes up through the circuit but into and through the pressure generator 300 itself exiting the orifice through which the pressure generator 300 typically draws air in from the environment. This has the effect of theoretically limiting retrograde exhaled volume to the volume of the conduit 400 and the pressure generator 300. However, given the typical volume contained within the conduit 400 and pressure generator 300, this scenario is unlikely to occur. Furthermore, as this results in the entirety of the circuit filling with the patient's exhaust, this fourth scenario can simply be considered a maximum possible example of the third scenario shown in FIG. 4.

As flow through the conduit 400 is the result of the pressure provided by the pressure generator 300, it should be understood that the above scenarios can also be discussed through the lens of pressure rather than air flow. In the first scenario shown in FIG. 2, the pressure generator 300 creates a pressure within the conduit 400 prior to the exhaust port 406 which is greater than or equal to the pressure provided by the patient's lungs 606. As such, none of the patient's exhalation is capable of traveling retrograde up the conduit 400. In the second scenario shown in FIG. 3, and to a greater extent in the third scenario shown in FIG. 4, the pressure generator 300 creates a pressure within the conduit 400 prior to the exhaust port 406 that is less than the pressure provided by the patient's lungs 606, and as such, the patient's exhalation is capable of traveling retrograde up the conduit 400. In one particular instance, the patient exhalation air flow 830 can be exactly equal to the maximum exhaust possible out of the exhaust port 406. Therefore the circuit air flow 810 will be zero, but there is still pressure within the conduit 400 prior to the exhaust port 406.

Likewise, it should be understood that the above scenarios can also be discussed through the lens of volume rather than air flow. In the first scenario shown in FIG. 2, the volume of air exiting the exhaust port 406 during the entirety of the patient's exhalation period is greater than or equal to the total volume of the patient's exhalation. As such, none of the patient's exhalation is capable of traveling retrograde up the conduit 400. Likewise, in the second scenario shown in FIG. 3, the volume of air exiting the exhaust port 406 during the entirety of the patient's exhalation period is still greater than or equal to the total volume of the patient's exhalation, even though there is some amount of retrograde flow. Because the total volume of air exiting the exhaust port 406 during the entirety of the patient's exhalation period exceeds the patient's exhaled volume, the entirety of the volume of retrograde flow is still exhausted before inhalation begins. However, in the third scenario shown in FIG. 4, the volume of air exiting the exhaust port 406 during the entirety of the patient's exhalation period is less than the total volume of the patient's exhalation, and, as such, the patient's exhalation which travels retrograde up the conduit 400 is not fully cleared before inhalation begins.

With deadspace rebreathing, the volume of deadspace is finite and thus the rebreathed gas is limited. A patient who experiences lower oxygen intake due to deadspace rebreathing may automatically increase their tidal volume to effectively reduce the negative effects of rebreathing. However, if the patient experiences circuit rebreathing, such as described in the third scenario shown in FIG. 4, the same response of increasing tidal volume will only exacerbate the problem as the larger associated exhaled volume will cause even greater retrograde flow, resulting in even greater rebreathing. This is a vicious cycle which, if it is allowed to continue, can make CPAP use both very uncomfortable and potentially dangerous.

Of course, for normal sized patients, the danger of circuit rebreathing is typically present at low pressure ranges, e.g., 4-6 cmH₂O, where exhaust flow is relatively low. However, for the many patients who use APAP (automatically titrated CPAP) and who have a very low starting pressure limit, the pressure starts at the low end of the range and settles where obstructive events are sufficiently eliminated. At the beginning of treatment, when the pressure is at the lowest possible setting, the risk of rebreathing is very high. Potentially, many patients in this situation experience rebreathing on a nightly basis. In addition to the deleterious physiological effects of rebreathing CO₂ discussed above, the discomfort associated with rebreathing likely results in many such patients abandoning the therapy that they likely very much need.

While leak in the mask (or any portion of the circuit between the patient and the exhaust port) clearly cannot address the circuit rebreathing discussed above, one possible solution to circuit rebreathing is simply to increase the maximum allowable flow through the exhaust port and/or to intentionally allow more leak in portions of the circuit between the pressure generator and the exhaust port. However, if a CPAP device were physically modified in this manner to ensure that there is no possibility of rebreathing, when high pressures are required (e.g., to counter obstructive events), there will be excessive exhaust flow. This can result in other problems that can make adherence difficult. One such issue is that large flows are very loud and potentially disruptive to sleep for both the patient and bed partner. Likewise, the presence of a large flow of air impinging on the patient or bed partner can also be disruptive to sleep. Additionally, large flows also quickly drain the fluid reservoir for humidification, thus potentially depriving the patient of the benefits of humidification throughout the night.

In view of the above considerations, the present invention is directed to methods for reducing CO₂ rebreathing caused by positive airway pressure machines.

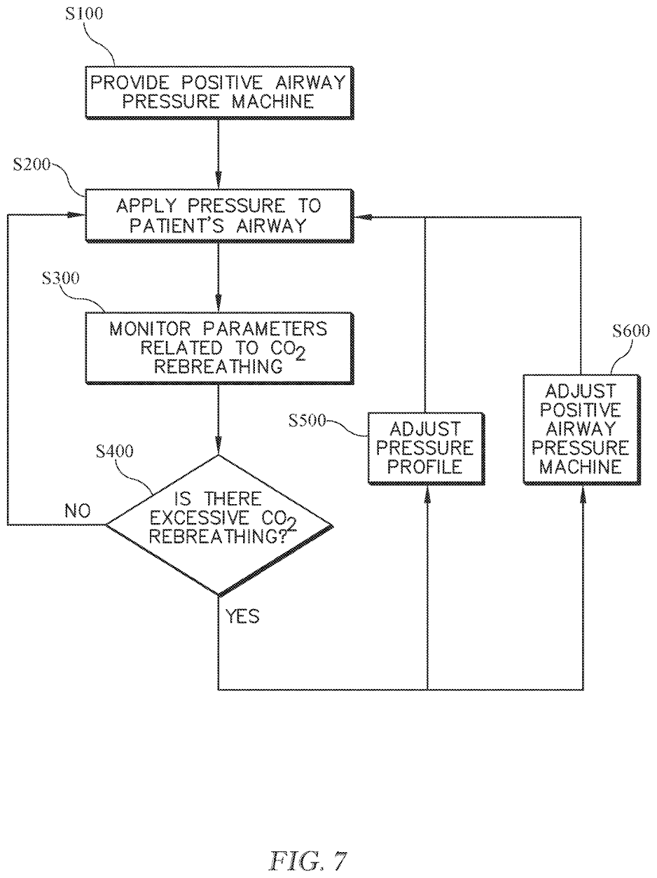

Referring now to FIG. 7, in one exemplary implementation of the present invention, in a step S100, a positive airway pressure machine (e.g., the positive airway pressure system 200 shown in FIGS. 1-4) is provided and a mask (e.g., the mask 700 shown in FIG. 5) is placed over a patient's face.

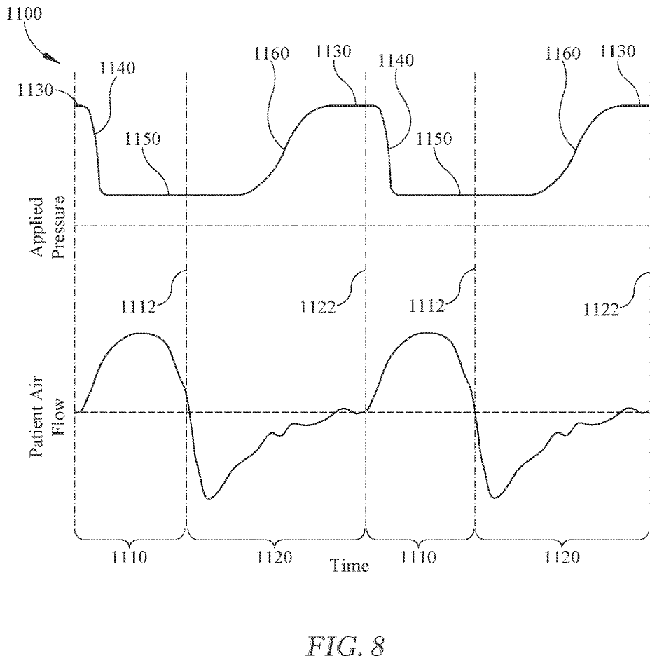

Next, in a step S200, pressure is provided into the patient's airway through the conduit 400 and mask 700 over a treatment period that includes a plurality of inhalation periods (e.g. as illustrated in FIG. 1) and a plurality of exhalation periods (e.g., as illustrated in the scenarios shown in FIGS. 2-4). In the most basic applications of positive airway pressure therapy, a continuous pressure is applied during both the inhalation period and the exhalation period, which is commonly referred to as continuous positive airway pressure (CPAP). In other known applications, there are two different pressures provided (BPAP). In one exemplary implementation of the present invention, and referring now to FIG. 8, throughout a patient's breath cycle, use of the positive airway pressure system 200 of the present invention results in a pressure profile 1100 that includes a relief positive airway pressure (RPAP) 1150 applied during a portion of the patient's inhalation periods 1110 including an end of the patient's inhalation period 1112 (i.e., a beginning of the next exhalation period 1120) and a therapy positive airway pressure (TPAP) 1130 applied during a portion of the exhalation periods 1120 including an end of the exhalation period 1122 (i.e., a beginning of the next inhalation period 1110). U.S. patent application Ser. No. 18/536,800, filed on Dec. 12, 2023 and entitled “POSITIVE AIRWAY PRESSURE SYSTEMS AND METHODS FOR TREATING SLEEP DISORDERED BREATHING,” provides additional description of pressure profiles that can be provided by the positive airway pressure system 200 of the present invention, and is thus incorporated herein by this reference.

Periodically during the treatment period, in a step S300, one or more parameters relating to the patient and/or the positive airway pressure machine are monitored, and in a step S400, it is determined whether the patient is experiencing excessive CO₂ rebreathing. For example, as discussed above, a certain amount of rebreathing is expected from deadspace rebreathing, and even some small amount of circuit rebreathing may be acceptable. By comparison, as used herein, “excessive CO₂ rebreathing” means any elevation of CO₂ beyond ambient CO₂ levels which results in a negative physiological effect to the patient. There is a direct association between the presence of dissolved CO₂ and pH, namely, increased CO₂ concentrations result in a drop in blood and tissue pH. Performance effects, particularly of the cognitive variety, manifest even at fairly low increases in CO₂ concentration above normal. For reference, normal atmospheric air has a concentration of about 0.04% (400 ppm or about 0.3 mmHg at sea-level), while arterial CO₂, PₐCO₂, is in the range of 35 to 45 mmHg partial pressure (46,000 ppm to 59,000 ppm). Table 1 below was taken from the Wisconsin Department of Health Services (https://www.dhs.wisconsin.gov/chemical/carbondioxide.htm) and illustrates various effects of routine daily exposure to varying levels of CO₂ exposure. Of note, a CO₂ concentration of 5,000 ppm is also the 8-hour time-weighted concentration limit set by OSHA.

TABLE 1

| CO₂ | |
|---|---|
| Concentration | |
| (ppm) | Condition and Effects |
| 400 | Normal atmospheric air |
| 400-1,000 | Enclosed room with good air exchange |
| 1,000-2,000 | Complaints of drowsiness and poor air |
| 2,000-5,000 | Headaches, sleepiness and stagnant/stale/stuff |
| | air - poor concentration, loss of attention, |
| | increase heart rate and slight nausea may be |
| | present |
| 5,000* | Indicates unusual air conditions - permissible |
| | daily exposure limit for workplace |
| 40,000+ | Level that is immediately harmful |

The current standard for sleep apnea device mask design guidelines (ISO 17510:2015—Medical devices—Sleep apnoea breathing therapy—Masks and application accessories) requires a maximum recommended time-weighted average for inspired CO₂ of 1% (10,000 ppm). Accordingly, in some exemplary implementations, “excessive CO₂ rebreathing” is defined as any rebreathing that results in inspired CO₂ of 1%.

However, as outlined in this ISO standard, “An inspired CO₂ fraction of 1% would add 1,013.25 Pa (7.6 torr) to the test model in Annex F and would result in the test end-tidal CO₂ value of 1,013.25 Pa (7.6 torr)+5,066.25 Pa (38 torr) or 6,079.5 Pa (45.6 torr). This represents a 20% increase in the CO₂ level. Based on this, the committee chose a 20% increase in the CO₂ level normal condition limit. Similarly, the 60% increase in the CO₂ level single fault condition limit represents a time-weighted average for an inspired CO₂ of 3%.”

In other words, the current ISO mask design guidelines permit 10,000 ppm (i.e., 1%) for nightly exposure, which is twice the allowable workplace limit shown in Table 1. Moreover, the test condition in ISO 17510 recommends a patient with 500 mL tidal volume at a breath rate of 15 brpm and an I:E of 1:2. If a patient breathes with a larger tidal volume, a faster breath rate or a more equal I:E, this problem can be exacerbated. Accordingly, in some exemplary implementations, “excessive CO₂ rebreathing” includes any rebreathing that results in inspired CO₂ of about 0.5% (5,000 ppm), about 0.4% (4,000 ppm), about 0.3% (3,000 ppm), about 0.2% (2,000 ppm), or about 0.1% (1,000 ppm). In some particular implementations, “excessive CO₂ rebreathing” includes as any rebreathing that results in inspired CO₂ of about 0.09% (900 ppm), about 0.08% (800 ppm), about 0.07% (700 ppm), about 0.06% (600 ppm), or about 0.05% (500 ppm). In one exemplary implementation, “excessive CO₂ rebreathing” includes any amount of inspired CO₂ above the ambient atmospheric levels of CO₂.

Regardless of what level of CO₂ inspiration is considered to be excessive CO₂ rebreathing, and referring still to FIG. 7, if it is determined that the patient is not experiencing excessive CO₂ rebreathing, the pressure profile applied in step S200 is allowed to continue. If, however, it is determined that there is excessive CO₂ rebreathing, in a step S500 the pressure profile is adjusted to reduce CO₂ breathing and/or in a step S600 the positive airway pressure system is adjusted to reduce CO₂ breathing.

As directly measuring the level of inspired CO₂ may, depending on the particular system, be difficult or impossible, with respect to step S300, a variety of different parameters may be monitored which are correlated to the level of inspired CO₂.

According to some exemplary implementations, the one or more parameter monitored include the volume of a patient's exhalation. Furthermore, according to some exemplary implementations, the volume of the patient's exhalation may be estimated based on one or more physiological parameters including, but not limited to, patient height; patient weight; patient BMI; patient gender; patient age; a measured breath rate; a measured I:E ratio; a measured tidal volume; a measured minute volume; a measured exhalation pressure produced by the patient's lungs; a measured exhalation flow produced by the patient's lungs; and a measured peak expiratory flow. The measured values can be determined based on a single measurement or in some preferred embodiments, based on a plurality of measurements taken over time, such as a rolling average.

In some exemplary implementations, and referring once again to FIGS. 2-4, the one or more parameters monitored include the volume of the patient's exhalation that travels retrograde into the conduit during an exhalation period. This measured volume can be determined directly by measuring flow through the conduit 400 or it can be determined by comparing one or more other measurements. For example, this volume can be estimated by comparing the patient's exhalation air flow 830 to the known maximum flow possible out of the exhaust port 406.

A majority of the patient's exhalation volume should exit through the exhaust port 406. As used herein, a “majority” is at least 90%. In some exemplary implementations, a “majority” is at least 91%, at least 92%, at least 93%, at least 94%, at least 95%, at least 96%, at least 97%, at least 98%, or at least 99%.

The sensors 502 shown in FIGS. 1-4 and discussed above can be used to measure the one or more parameters. For example, flow meters positioned on or near the conduit 400 and mask 700 can measure the respective circuit air flow 810 and patient exhalation air flow 830. The particular sensors used and their placement is readily understood by one skilled in the art to allow for the measurement of the parameters discussed above.

Regardless of the particular parameters measured or the means of measurement, the purpose is to determine whether any of the patient's exhalation is present in the conduit at the beginning of each inhalation period. Advantageously, due to the bolus air flow through the conduit discussed above, this determination can be made without having to consider mixture of the exhaled gas with fresh gas.

In some exemplary implementations, in step S400, it is determined that the patient is not experiencing excessive CO₂ rebreathing when there is none of the patient's exhalation present in the conduit 400, i.e., the scenario shown in FIGS. 2 and 3. Of course, even when the exhalation air flow 830 is less than the exhaust flow 840, a small amount of the patient's exhalation may travel retrograde up into the conduit 400 due merely to mixing of the exhalation air flow 830 and circuit air flow 810. This trace amount of exhalation can easily be ignored as it will have a negligible effect on the patient.

According to one exemplary implementation, in a step S500, the pressure profile applied by the positive airway pressure system is adjusted to reduce CO₂ breathing. In particular, the pressure provided by the pressure generator 300 is adjusted such that the majority of the patient's exhalation is directed out of the exhaust port 406. Likewise, the pressure provided by the pressure generator 300 is adjusted such that a volume of air exiting the exhaust port is greater than or equal to the volume of the patient's exhalation.

For example, in some exemplary implementations, when it is determined that the patient is experiencing excessive CO₂ rebreathing, the pressure provided by the pressure generator 300 is increased until the patient no longer experiences excessive CO₂ rebreathing.

In some exemplary implementations, the pressure is increased only at an end portion of the exhalation period in order to clear the patient's exhalation from the conduit 400 via the exhaust port 406.

Preferably, the patient's exhalation is cleared from the conduit prior to the beginning of the next inhalation period so as to avoid any circuit rebreathing. However, it is also contemplated that in some implementations, at a beginning portion of the next inhalation period, there is still at least some of the patient's exhalation within the conduit 400. According to these implementations, the pressure provided by the pressure generator 300 is kept at an elevated level or raised until the patient's exhalation is cleared from the conduit 400 via the exhaust port 406. However, it is preferred that during the beginning portion of the next inhalation period, the patient still does not experience excessive CO₂ rebreathing.

In any event, once the patient's exhalation is cleared from the conduit 400, in some embodiments, the pressure is decreased.

The particular methods of increasing and decreasing the pressure are not limited. In some embodiments, the pressure may be increased as soon as it is determined that circuit rebreathing will occur, e.g., once it is determined that a sufficient volume of the patient's exhalation has traveled retrograde up into the conduit 400. In some other embodiments, the pressure is increased during the next exhalation period so that the patient may experience one breath with excessive CO₂, but is thereafter no longer experiencing excessive CO₂ rebreathing. In still other embodiments, the pressure is gradually increased over two or more subsequent exhalation periods. Although this may result in slightly more CO₂ rebreathing, the gradual increase in pressure is less likely to be noticed by the patient.

Furthermore, a factor of safety may be applied so that fluctuations in the patient's respiration do not result in occasional CO₂ rebreathing. In particular, a running average of the patient's exhaled volume can be calculate with the pressure adjusted so that the exhaust volume is equal to the average patient exhaled volume plus a factor of safety. In some embodiments, this factor of safety is about 20% while in other embodiments, the factor of safety is about 15%, about 10%, or about 5%. While this may result in slightly higher pressure than is necessary to prevent excessive CO₂ rebreathing, it is contemplated that it will result in a more consistent application of pressure over time, which may ultimately increase patient comfort.

Further still, according to some exemplary implementations of the present invention, the pressure provided by the pressure generator 300 can take into account any intentional or unintentional leak. When there is retrograde flow of the patient's exhalation, it does not matter whether this is cleared through the exhalation valve, through intentional leak of the mask, or through unintentional leak, so long as it is no longer present at the beginning of the next inhalation. For example, it is well known that with systems that use a full face mask, patients with beards experience higher levels of leak than patients with little or no facial hair. However, the specific level of unintentional leak varies from patient to patient, or even possibly from night to night. Accordingly, in some implementations of the present invention, unintentional leak is measured and the pressure provided by the pressure generator 300 is correspondingly reduced. In other words, exhaust flow is not necessarily limited only to flow through the exhaust port 406 shown in FIGS. 1-4 but may be inclusive of any leak, whether intentional or unintentional.

According to one exemplary implementation, in a step S600, the positive airway pressure system is physically tuned to reduce CO₂ rebreathing. In some exemplary implementations, an exhaust assembly and/or mask of the positive airway pressure system can be replaced with an alternative version that provides a predetermined maximum exhaust flow. In one other exemplary implementation, and referring once again to FIGS. 5-6, the insert 730 included within the secondary outlet 726 of the exhaust assembly 720 can be replaced with one of a plurality of interchangeable ports that each provide a different predetermined maximum exhaust flow out of the secondary outlet 726. While these particular methods of tuning the positive airway pressure system can't easily be accomplished immediately or dynamically during a treatment period, tuning the positive airway pressure system in this manner does provide gross adjustments of the machine. It is contemplated that in some exemplary implementations, the positive airway pressure system provides feedback to the user regarding what adjustments are needed. For example, the system could instruct the user to the specific interchangeable port required to reduce further CO₂ rebreathing.

In some other exemplary embodiments, the exhaust assembly has an adjustable exhaust port. For example, although not expressly shown, the exhaust port may define an exhaust orifice with an adjustable size which can be physically changed. There are several means of adjusting the size of an exhaust orifice well known in the art and which can readily be incorporated into the exhaust assembly of the present invention, but to provide a non-limiting example, in one particular embodiment, two plates which each define one or more holes can be rotated relative to one another to selectively align the holes and adjust the overall orifice defines by the exhaust orifice. In some exemplary implementations, the positive airway pressure system includes mechanisms to actively control the adjustable exhaust port.

Referring once again to FIG. 8, as previously mentioned, in one exemplary implementation of the present invention, use of the positive airway pressure system 200 of the present invention results in a pressure profile 1100 that includes a relief positive airway pressure (RPAP) 1150 applied during a portion of the patient's inhalation periods 1110 including an end of the patient's inhalation period 1112 (i.e., a beginning of the next exhalation period 1120) and a therapy positive airway pressure (TPAP) 1130 applied during a portion of the exhalation periods 1120 including an end of the exhalation period 1122 (i.e., a beginning of the next inhalation period 1110). As shown in FIG. 8, the TPAP 1130 is greater than the RPAP 1150. Furthermore, the pressure decreases from the TPAP 1130 to the RPAP 1150 through a relief transition (RT) 1140 that occurs during an early portion of the inhalation period 1110 (i.e., after the end of the exhalation period 1120). Similarly, the pressure increases from the RPAP 1150 to the TPAP 1130 through a therapy transition (TT) 1160 that occurs partway through the exhalation period 1120.

According to some exemplary implementations of the present invention in which the positive airway pressure system 200 provides a pressure profile 1100 shown in FIG. 8, when it is determined in step S400 that the patient is experiencing excessive CO₂ rebreathing, in step S500, the RPAP 1150 is increased. Increasing the pressure during RPAP 1150, and especially the portion of RPAP 1150 that occurs during the exhalation period 1120, will help clear the conduit of the patient's exhalation prior to the next inhalation period.

Similarly, in other exemplary implementations, when it is determined in step S400, that the patient is experiencing excessive CO₂ rebreathing, in step S500, the TPAP 1130 is increased. Increasing the pressure during TPAP 1130, and especially the portion of TPAP 1130 that occurs during the exhalation period 1120, will help clear the conduit of the patient's exhalation prior to the next inhalation period.

Rather than adjust the level of pressure provided, in other exemplary implementations the timing of TT and/or RT is adjusted.

In some exemplary implementations, when it is determined in step S400, that the patient is experiencing excessive CO₂ rebreathing, in step S500, the timing of the TT 1160 is adjusted to occur earlier during the exhalation period 1120. As such, the higher TPAP 1130 is maintained for a larger portion of the exhalation period 1120 which will help clear the conduit of the patient's exhalation.

In some exemplary implementations, when it is determined in step S400, that the patient is experiencing excessive CO₂ rebreathing, in step S500, the timing of the RT 1140 is adjusted to occur later during the inhalation period 1110. Accordingly, while there is still at least some of the patient's exhalation within the conduit 400 at a beginning portion of the next inhalation period, the higher TPAP 1130 is maintained until the patient's exhalation is cleared from the conduit 400 via the exhaust port 406.

The adjustments to the pressure profile discussed above are merely exemplary, and it should be understood that they can be utilized alone or in combination.

While FIG. 8 shows only two breath cycles of an inhalation period 1110 and exhalation period 1120, it should be understood that this is merely exemplary and that the methods described above occur over a period that includes a plurality of inhalation periods and a plurality of exhalation periods. Furthermore, although the pressure profile 1100 shown in FIG. 8 repeats identically between each breath cycle of an inhalation period 1110 and exhalation period 1120, it is contemplated that in some exemplary implementations, one or more of the TPAP 1130, the RT 1140, the RPAP 1150, and the TT 1160 may vary between breath cycles. Some of these potential changes have been discussed above, but additional details can be found in U.S. patent application Ser. No. 18/536,800 regarding pressure profiles that can be provided by the positive airway pressure system 200 of the present invention. The adjustments made to the pressure profile in step S500 described above can be implemented individually or in combination with each of the various embodiments described in U.S. patent application Ser. No. 18/536,800 with respect to the TPAP, RT, RPAP and/or TT.

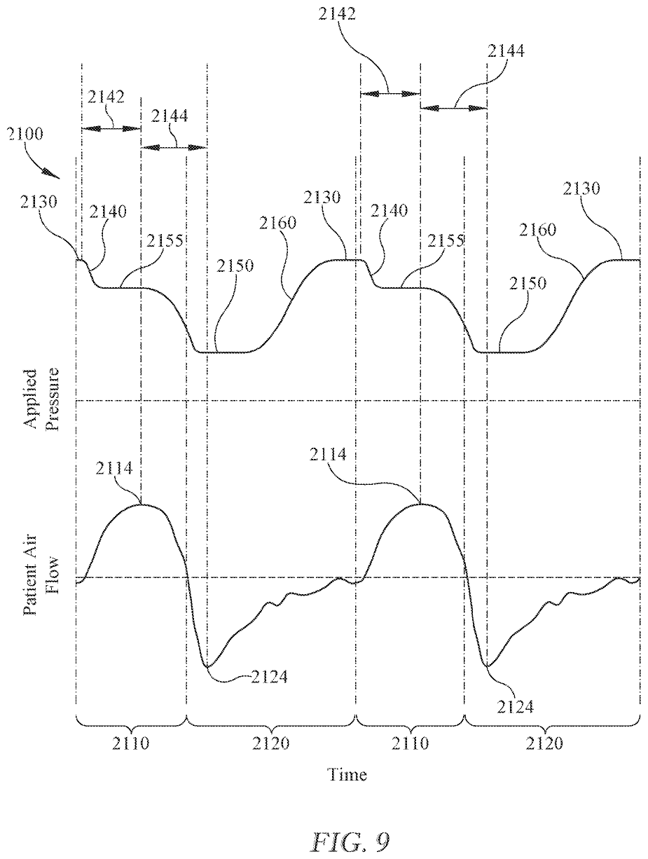

For example, and referring now to FIG. 9, in some exemplary implementations, the relief transition can occur in a plurality of steps to further enhance comfort. As shown in FIG. 9, and similar to the implementation show in FIG. 8, in the pressure profile 2100 of this exemplary implementation, the pressure decreases from the TPAP 2130 to the RPAP 2150 through a RT 2140 that begins shortly after the beginning of the inhalation period 2110 but still during an early portion of the inhalation period 2110. Similarly, the pressure increases from the RPAP 2150 to the TPAP 2130 through a TT 2160 that occurs partway through the exhalation period 2120. However, unlike the implementation shown in FIG. 8, during the RT 2140 shown in FIG. 9, there is an initial decrease in pressure during a first period of time 2142, which is followed by a subsequent decrease in pressure during a second period of time 2144.

During the first period of time 2142, the pressure provided within the patient's airway decreases from the TPAP 2130 an initial amount before maintaining substantially constant at an intermediate pressure 2155 that is less than TPAP 2130 but greater than RPAP 2150. In this exemplary implementation shown in FIG. 9, the first period of time 2142 ends when the patient's air flow reaches a peak inhalation flow 2114 while the second period of time 2144 ends when the patient's air flow reaches a peak exhalation flow 1124. As such, the overall period of time over which the RT 2140 occurs in FIG. 9 is significantly longer than the RT 1140 shown in FIG. 8. However, in other implementations, the first drop in pressure from TPAP 1130 to the intermediate pressure 2155 is done relatively quickly and the second drop in pressure from the intermediate pressure 2155 to RPAP 2150 occurs at or after the peak inhalation flow 2114. Other timings for the first drop and the second drop are also possible without departing from the spirit and scope of the present invention. Furthermore, although only two drops in pressure are shown during the RT 2140 in FIG. 9, it is contemplated that three or more drops can occur without departing from the spirit and scope of the present invention.

The TPAP 2130, RT 2140, RPAP 2150, and TT 2160 of the pressure profile 2100 shown in FIG. 8 can be modified in a similar manner as the TPAP 1130, the RT 1140, the RPAP 1150, and the TT 1160 described above with reference to FIG. 8.

However, according to some exemplary implementations of the present invention in which the positive airway pressure system 200 provides a pressure profile 2100 shown in FIG. 9, when it is determined in step S400 that the patient is experiencing excessive CO₂ rebreathing, in step S500, the multiple drops in pressure during RT 2140 can also be modified individually. For example, a first drop in pressure may still occur, but one or more subsequent drops in pressure may be removed when it is determined that the patient is experiencing excessive CO₂ rebreathing. This will result in an overall increase in RPAP 2150, which as previously noted will help clear the conduit of the patient's exhalation prior to the next inhalation period.

It should further be appreciated that the methods described above can also vary depending on whether the patient is awake or asleep, or even some state in-between. Generally, comfort has priority while a patient is awake and therapy has priority while a patient is asleep. While the patient is awake, i.e., when comfort is prioritized, it is important to keep the pressure lower. However, there still should be sufficient pressure to clear the conduit of the patient's exhalation prior to the next inhalation period. Once the patient falls asleep, the degree of comfort required only needs to be maintained such that the patient is not aroused from sleep, so the pressure can be increased.

Accordingly, in some exemplary embodiments, the positive airway pressure system 200 is capable of determining whether the patient is awake or asleep and changing one or more parameters of the pressure profile accordingly. For example, the pressure may be lowered to a minimum pressure while the patient is awake or in light sleep and then augmenting the pressure gradually over a ramping period of time as the patient is asleep, reaches deeper sleep, and/or is asleep for longer. For example, in some exemplary implementations, the process of augmenting the pressure includes a gradual increase of the pressure provided at the end of the exhalation period (e.g., the TPAP 1130 shown in FIG. 8 increases) over successive exhalation periods.

In some exemplary implementations, the process of augmenting the pressure includes a gradually longer period of time that a higher pressure is provided at the end of the exhalation period (e.g., the TT 1160 occurs sooner during the exhalation period 1120 shown in FIG. 8) over successive exhalation periods. By beginning TT 1160 earlier in the exhalation period 1120, there is more time to gradually transition from RPAP 1150 to TPAP 1130 while still providing the necessary pressure over time to clear the conduit of the patient's exhalation prior to the next inhalation period. Means of determining whether the patient is awake or asleep include, but are not limited to, detecting rapid eye movement (REM); detecting flow limitation or increased upper airway resistance; detecting a response to central sleep apnea; and monitoring respiratory parameter variance (e.g., breath rate, tidal volume, peak flows, etc.) since breathing generally becomes regular once a patient transitions to being asleep. Measurements used to determine whether the patient is awake or asleep can be performed by sensors or mechanisms integrated to the positive airway pressure machine or they can be provided as a separate or external piece of equipment configured to transmit data to the positive airway pressure machine.

Of course, a patient's breathing is not always steady throughout the night, even if their sleep is not interrupted. As such, it is important to monitor the patient's exhalation levels and adjust the pressure as needed to reduce, and preferably eliminate, circuit rebreathing. To this end, in some exemplary implementations, an initial pressure is first provided during a first exhalation period which is sufficiently high to guarantee that none of the patient's exhalation travels retrograde into the conduit. The pressure provided during successive exhalation periods is then decreased until a minimal amount of circuit rebreathing occurs. The pressure is then increased until none of the patient's exhalation travels retrograde into the conduit. In some embodiments, the amount the pressure is decreased over the successive exhalation periods is set at a predetermined amount and the final increase in pressure is performed at a level that is less than or equal to this predetermined amount.

This step of titrating the pressure may occur continuously throughout a treatment period or be repeated periodically throughout the treatment period. In any event, the sensors 502 discussed above with respect to FIG. 1 can be used to monitor the one or more parameters to determine whether a portion of the patient's exhalation travels retrograde into the conduit as discussed above.

Furthermore, according to some exemplary implementations, by continuously monitoring the parameters discussed above, it is possible to calculate mask leak characteristics over time. This can then be used to compare the actual mask leak to an expected amount of leak. If the actual mask leak falls below a minimum threshold, it can indicate that a problem is occurring with the mask itself, such as partial occlusion of one or more leak ports with dirt and/or condensation. Accordingly, in some implementations of the present invention, if the calculated mask leak is below a minimum threshold, a warning is provided to the patient. In some implementations, feedback is provided that some portion of the CPAP device should be changed such as the leak port, the mask itself, and/or some other portion of the circuit. It has bene recognized that, in some situations, replacing the conduit 400 can also improve circuit rebreathing. A smaller diameter tube will result in faster flow speeds which clears the circuit more quickly.

One of ordinary skill in the art will recognize that additional embodiments and implementations are also possible without departing from the teachings of the present invention or the scope of the claims which follow. This detailed description, and particularly the specific details of the exemplary embodiments disclosed herein, is given primarily for clarity of understanding, and no unnecessary limitations are to be understood therefrom, for modifications will become apparent to those skilled in the art upon reading this disclosure and may be made without departing from the spirit or scope of the claimed invention.

What is claimed is:

1. A method for reducing CO₂ rebreathing from a positive airway pressure machine including a pressure generator, a mask configured for placement over a patient's face, a conduit operably connecting the pressure generator to the mask, and an exhaust port, the method comprising:
providing pressure into the patient's airway through the conduit and mask over a treatment period that includes a plurality of inhalation periods and a plurality of exhalation periods;
wherein the pressure provided is at a level sufficient to direct a majority of the patient's exhalation out of the exhaust port.

2. The method of claim 1, wherein, during each inhalation period, the patient inhales less than 0.1% CO₂.

3. The method of claim 1, wherein, at the beginning of each inhalation period, none of the patient's exhalation is present in the conduit.

4. The method of claim 1, further including the steps of:
measuring a volume of the patient's exhalation; and
adjusting the pressure provided such that the majority of the patient's exhalation is directed out of the exhaust port.

5. The method of claim 1, further including the steps of:
determining that a portion of the patient's exhalation travels into the conduit during an exhalation period; and
augmenting the pressure to clear the patient's exhalation from the conduit via the exhaust port.

6. The method of claim 5, wherein augmenting the pressure includes increasing the pressure provided at an end portion of the exhalation period.

7. The method of claim 5, wherein augmenting the pressure includes increasing the time during an exhalation period when an increased pressure is provided.

8. The method of claim 5, further including a step of decreasing the pressure provided after the patient's exhalation is cleared from the conduit via the exhaust port.

9. The method of claim 5, wherein augmenting the pressure includes increasing the pressure provided during a beginning portion of the next inhalation period until the patient's exhalation is cleared from the conduit via the exhaust port.

10. The method of claim 1, further including the steps of:
providing an initial pressure during a first exhalation period, the initial pressure configured such that none of the patient's exhalation travels into the conduit;
decreasing the pressure provided during successive exhalation periods by a predetermined amount until a portion of the patient's exhalation travels into the conduit; and
increasing the pressure to the last pressure when none of the patient's exhalation traveled into the conduit.

11. The method of claim 1, further including the steps of:
receiving data from one or more sensors that measure the pressure or flow through the conduit; and
determining whether a portion of the patient's exhalation travels into the conduit.

12. The method of claim 1, further including the steps of:
determining whether the patient is awake or asleep;
providing a minimum pressure when the patient is awake; and
augmenting the pressure provided over a ramping period of time after the patient is asleep.

13. The method of claim 12, wherein the minimum pressure is sufficient to ensure that, at the beginning of an inhalation period, none of the patient's exhalation is present in the conduit.

14. The method of claim 12, wherein, during the step of augmenting the pressure, the pressure provided at an end of each exhalation period is gradually increased over successive exhalation periods.

15. The method of claim 12, wherein the pressure provided increases during each exhalation period to reach a higher pressure at a period of time prior to the next inhalation period, and during the step of augmenting the pressure, the higher pressure is gradually decreased over successive exhalation periods.

16. The method of claim 12, wherein the pressure provided increases during each exhalation period to reach a higher pressure at a period of time prior to the next inhalation period, and during the step of augmenting the pressure, the period of time is gradually increased over successive exhalation periods.

17. The method of claim 1, wherein the pressure is at a relief positive airway pressure during a portion of the inhalation periods including an end of the inhalation period;
wherein the pressure is at a therapy positive airway pressure during a portion of the exhalation periods including an end of the exhalation period, the therapy positive airway pressure greater than the relief positive airway pressure; and
wherein the pressure decreases from the therapy positive airway pressure to the relief positive airway pressure after the end of the exhalation period.

18. The method of claim 17, further including the steps of:
determining that a portion of the patient's exhalation travels into the conduit during an exhalation period; and
increasing the relief positive airway pressure.

19. The method of claim 17, further including the steps of:
determining that a portion of the patient's exhalation travels into the conduit during an exhalation period; and
maintaining the therapy positive airway pressure for a larger portion of the exhalation periods.

20. The method of claim 17, further including the steps of:
determining that a portion of the patient's exhalation travels into the conduit during an exhalation period; and
increasing the therapy positive airway pressure.

21. The method of claim 1, further including a step of modifying the exhaust port to adjust a maximum exhaust flow out of the exhaust port.

22. The method of claim 21, wherein the step of modifying the exhaust port includes replacing the exhaust port with one of a plurality of interchangeable ports, each of the interchangeable ports having a predetermined exhaust flow rate.

23. The method of claim 21, wherein the exhaust port includes an exhaust orifice with an adjustable size and the step of modifying the exhaust port includes changing the size of the exhaust orifice.

24. The method of claim 21, further including a step of providing feedback to a user regarding what modification to the exhaust port is needed.

25. A method for reducing CO₂ rebreathing from a positive airway pressure machine including a pressure generator, a mask configured for placement over a patient's face, a conduit operably connecting the pressure generator to the mask, and an exhaust port, the method comprising:
providing pressure into the patient's airway through the conduit and mask over a treatment period that includes a plurality of inhalation periods and a plurality of exhalation periods;
measuring a volume of the patient's exhalation; and
adjusting the pressure provided at an end portion of an exhalation period such that none of the patient's exhalation is present in the conduit at a beginning of an inhalation period.

26. A method for reducing CO₂ rebreathing from a positive airway pressure machine including a pressure generator, a mask configured for placement over a patient's face, a conduit operably connecting the pressure generator to the mask, and an exhaust port, the method comprising:
measuring a volume of the patient's exhalation;
providing pressure into the patient's airway through the conduit and mask over a treatment period that includes a plurality of inhalation periods and a plurality of exhalation periods, wherein, during a first exhalation period, the pressure is provided at an initial pressure configured such that none of the patient's exhalation travels into the conduit;
decreasing the pressure provided during successive exhalation periods by a predetermined amount until a portion of the patient's exhalation travels into the conduit; and
increasing the pressure to the last pressure when none of the patient's exhalation traveled into the conduit.
